(19) United States
(12) Patent Application Publication
(10) Pub. No.: US 2017/0014719 A1
(43) Pub. Date: Jan. 19, 2017

(54) SYSTEM AND METHOD FOR SUPER-REALITY ENTERTAINMENT

(71) Applicant: Takayuki Arima, Tokyo (JP)

(72) Inventor: Takayuki Arima, Tokyo (JP)

(21) Appl. No.: 15/279,793

(22) Filed: Sep. 29, 2016

Related U.S. Application Data
(63) Continuation-in-part of application No. 13/481,618, filed on May 25, 2012, now abandoned.

(51) Int. Cl. A63F 13/655; H04N 13/00; H04N 13/02; G06F 3/01; A63F 13/816; H04R 5/027; A63F 13/46; A63F 13/215; A61B 5/025; H04N 5/232; H04R 3/00

(52) U.S. Cl. CPC A63F 13/655; H04N 5/23267; H04N 13/0007; H04N 13/0203; H04N 13/0282; G06F 3/011; H04R 3/005; H04R 5/027; A63F 13/46; A63F 13/215; A61B 5/025; A63F 13/816; A63F 2300/8082; A63F 2300/8029

(57) ABSTRACT

A system and method are configured to provide a user with entertainment, for which one or more activities are prepared, each activity involving one or more participants. Specifically, the system and method are configured to obtain user information including the user's selection of an activity and a participant in the activity, receive images and sounds captured by at least one camera and one microphone attached to the participant, process the images and sounds, send the processed images and sounds to a client terminal associated with the user who selected the activity and the participant in the activity, and provide the user with a game based on a process of the activity.

### CROSS REFERENCE

This application is a continuation-in-part of U.S. patent application Ser. No. 13/481,618, filed on May 25, 2012.

### BACKGROUND

As new generations of smart phones, laptops, tablets and other wireless communication devices are embedded with increased number of applications, users are increasingly demanding to obtain high quality experiences with the applications particularly in the mobile entertainment arena. An application herein refers to a computer program designed to help users perform tasks via the operation system in the device. Such applications include programs for video viewing, digital media downloading, games, navigations and various others. In recent years, reality TV shows and variety shows including games, cooking contests, singing contests and various other entertaining events are becoming popular, indicating the current trend of viewers' preference. However, participants in a reality TV show, for example, are often persuaded to act in specific scripted ways by off-screen producers, with the portrayal of events and speech highly manipulated. Furthermore, a traditional way of viewing variety shows, sports, documentaries, performing arts, etc. gives viewers with a sense of merely observing them as a spectator.

The present invention is directed to a new type of entertainment system and method that enable users to enjoy viewing as well as games based on vivid images and sounds as perceived by a participant in an actual activity. Examples of such activities may include: adventure, sport, vacationing, contest, etc. Such entertainment can provide the viewer with the realistic sensation filled with on-site and unexpected excitements, compounded with the activity-based game, thereby opening up a new entertainment paradigm, which is referred to as “super-reality entertainment” herein in this document.

### BRIEF DESCRIPTION OF THE DRAWINGS

FIG. 1A illustrates an example of positions of cameras and microphones on a pair of goggles, worn by a mountain climber.

FIG. 1B illustrates an example view perceived by the mountain climber provided with the devices.

FIG. 2 illustrates an example of a system for providing super-reality entertainment.

FIG. 3 is a block diagram illustrating the management system.

FIG. 4 is a flowchart illustrating a method of providing a user with super-reality entertainment including a game based on a process of an activity.

FIG. 5 is a flowchart illustrating steps of executing the game.

FIG. 6 illustrates an example of a view displayed on a screen of a client terminal.

FIGS. 7A and 7B illustrate examples of the game information for the case of climbing Mt. XYZ with the elevation 4000 m.

### DETAILED DESCRIPTION

A system and method to achieve and manage the “super-reality entertainment” are described below with reference to accompanying drawings.

A football is an example of an activity in which the players can experience a high degree of excitement, fun and dynamics. The excitement among the players in such a collision sport is apparent owing to the real-time dynamics, involving rushing, kicking, tackling, intercepting, fumbling, etc. It is generally difficult for mere spectators to feel such excitements and sensations felt by the actual players. In a conventional broadcasting system, one or more cameras are provided at fixed locations outside the field where the activity takes place, providing views and sounds as perceived by a mere spectator at the location where the camera is placed. In contrast, enabling users to receive the vivid images and sounds as perceived by the actual participant can provide the excitement and sensation similar to what is felt by the participant himself/herself. Such entertainment may be realized by using a system that is configured to capture images and sounds as perceived by a participant in the activity, and transmit them to a user so that the user can realistically experience the activity as if he/she is participating in the activity. The system can be configured to allow each user to select an activity to view and a participant to get connection with for receiving the images and sounds captured by the participant during the activity. The sense of being bonded with the user's favorite participant/player can further enhance the enjoyment.

The case of playing football is mentioned above as an example. Obviously, there are many activities that people wish to participate in, but they normally give up doing so simply because they cannot afford to spend money or time, or they are scared or not physically fit to try. Enabling a user to receive the vivid images and sounds as perceived by a participant can provide the user with the exciting moments that the user would never experience otherwise. Such entertainment can be made available to users at minimal cost through the use of a TV broadcasting system, a desktop computer, or an application that is configured to run on smart phones, laptops, tablets or other mobile devices, in communication with a controlling program in a computer system or server. In most activities, one or more cameras and one or more microphones can be attached to or integrated with a head gear, a helmet, a hat, a headband, goggles, glasses or other item that the participant wears or directly attached to the head or face of the participant during the activity. Examples of such activities that users can enjoy by receiving the captured images and sounds may include, but not limited to, the following:
Mountain climbing by receiving images and sounds as perceived by a mountain climber.
Deep sea exploration by receiving images and sounds as perceived by a deep sea diver.
Spacewalk, moving in zero-gravity, walking on the moon and other space activities by receiving images and sounds as perceived by an astronaut.
Paranormal experience by receiving images and sounds as perceived by a so-called ghost hunter searching a haunted house.
Cave exploration by receiving images and sounds as perceived by a cave explorer.
Vacationing in an exotic location by receiving images and sounds as perceived by a vacationer.
Observing life and people in an oppressed or troubled country by receiving images and sounds as perceived by a reporter.
Sports by receiving images and sounds as perceived by an athlete, such as soccer, football, boxing, fencing, wrestling, karate, taekwondo, tennis and others.
Exploration to the North Pole or the South Pole by receiving images and sounds as perceived by an explorer.
Firefighting by receiving images and sounds as perceived by a firefighter.
Medical operation by receiving images and sounds as perceived by a surgeon.
Cooking by receiving images and sounds as perceived by a chef or an amateur.
Performing on stage by receiving images and sounds as perceived by a singer or an actor on stage.
Cleaning and processing garbage by receiving images and sounds as perceived by a cleaning crew member.
Encountering wild animals in Africa by receiving images and sounds as perceived by a traveler.
Crime scene investigation by receiving images and sounds as perceived by an investigator or a police officer.
Bad weather experience by receiving images and sounds as perceived by a tornado chaser.
Hot air balloon ride by receiving images and sounds as perceived by a rider.
Bungee jumping by receiving images and sounds as perceived by a jumper.

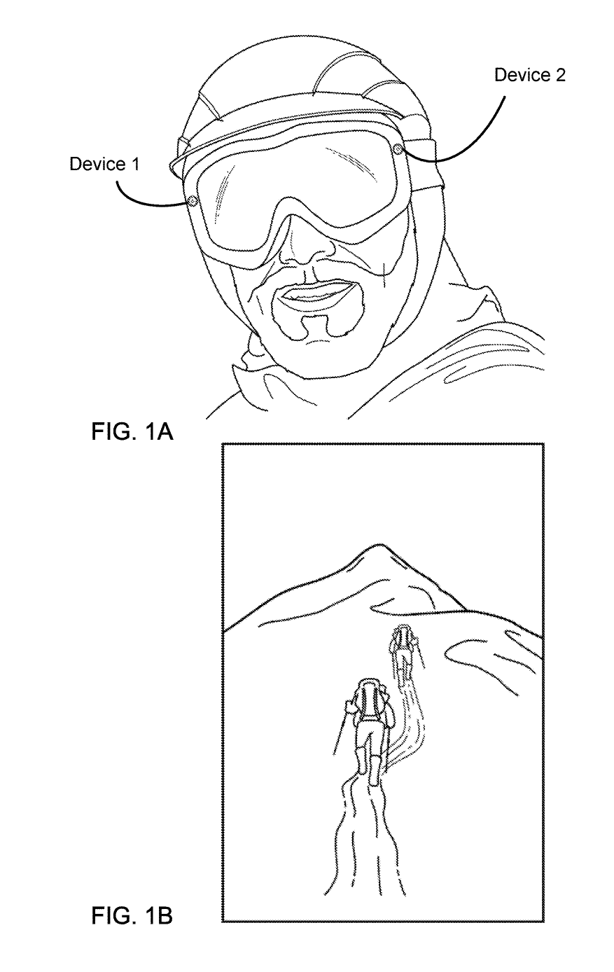

The images and sounds perceived by a participant in an activity can be captured by at least one camera and one microphone provided preferably in the proximity of his/her eyes and ears. FIG. 1A illustrates an example of positions of cameras and microphones on a pair of goggles, worn by a mountain climber, for example. In this example, a device including both a camera and a microphone integrated therein is used, and two such devices, device 1 and device 2, are attached to both sides of the goggles near the temples of the mountain climber who wears the goggles. One or more devices may be attached to a head gear, a helmet, a hat, a headband, goggles, glasses or any other head/face-mounted article, or may be directly attached to the bead or face of a participant during an activity. Two or more cameras can capture the images as seen from two or more perspectives, respectively, which can be processed by using a suitable image processing technique for the viewer to experience the 3D effect. Similarly, two separate microphones may be placed near the ears of the participant, to capture the sounds from two audible perspectives, respectively, which can be processed by using a suitable sound processing technique for the viewer to experience the stereophonic effect. In another example, a microphone may be placed at the back side of the head so that the sound from behind can be clearly captured to sense what's going on behind the participant in the activity. In the example illustrated in FIG. 1A, a device including both a camera and a microphone integrated therein is used, and two such devices are placed near the participant's temples at locations as close as possible to his eyes and ears. One or more devices, each including at least one camera and at least one microphone can be provided with each participant. Alternatively, at least one camera and at least one microphone individually can be provided with the participant. FIG. 1B illustrates an example view perceived by the mountain climber provided with such devices, as illustrated in FIG. 1B, wherein the backs of two preceding fellow mountain climbers and the summit of the mountain are in the view.

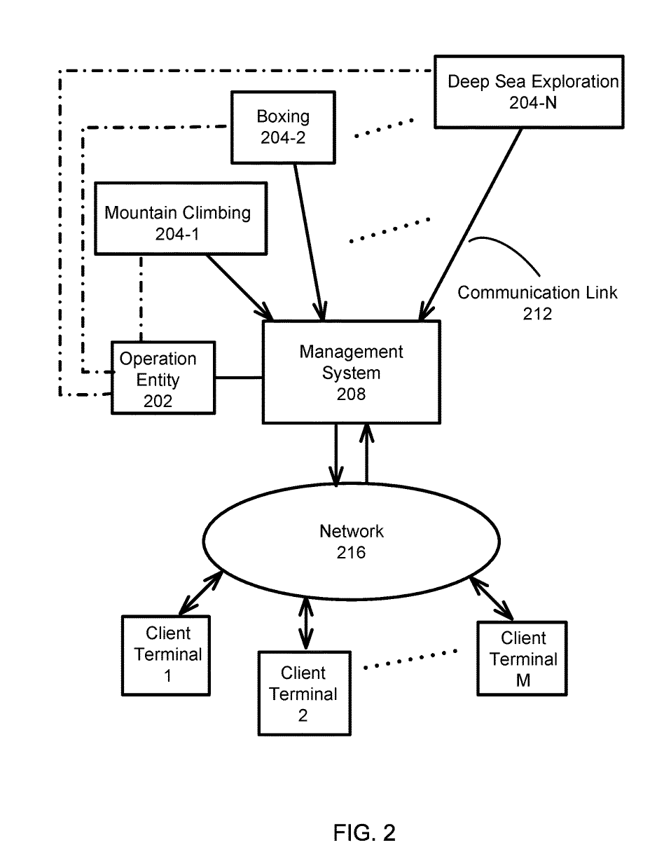

FIG. 2 illustrates an example of a system for providing super-reality entertainment by capturing images and sounds as perceived by a participant in an activity, and transmitting them to a user so that the user can realistically experience the activity as if he/she is participating in the activity. An operation entity 202 represents an organization, a company, a team or a person, who plans and manages the operation of the activities. For example, a number of activities of interest can be planned and prepared by the operation entity 202, as indicated by dashed-dotted lines in FIG. 2. The operation entity 202 may decide on the types of activities to pursue, schedule an activity to take place at a certain time and date, select a place that is proper for pursuing an activity, etc. Furthermore, the operation entity 202 may hire or contract with people who can actually participate in the activities, for example, an experienced mountain climber for mountain climbing 204-1, a professional boxer for boxing 2042-2, . . . and a diver with a biology background for deep sea exploration 204-N. The operation entity 202 may be further configured to pay for expenses to pursue the activities, such as travel expenses and equipment purchase/rental fees, in addition to paying wages to the participants and other supporting staff. Once the activity is planned, the operation entity 202 provides at least one of the one or more participants in the activity with at least one camera and one microphone to be attached to his/her head gear, helmet, hat, headband, goggles, glasses or other item that the participant wears or directly to the head or face of the participant. Thereafter, the planned activity is carried out at a predetermined time and place.

The number of cameras and the number of microphones provided with a participant may vary according to predetermined needs for image and sound reception. As mentioned earlier, a device including both a camera and a microphone, or other sensing devices may be used alternatively to using separate cameras and microphones. The vivid images and sounds captured by the participants in each activity are transmitted to a management system 208 through a communication link 212. The management system 208 is associated with and may be configured by the operation entity 202. The communication link 212 may represent a signal channel based on wireless communication protocols, satellite transmission protocols, or any other signal communication schemes.

The management system 208 may be located in a server and is configured to receive and process the signals including the images and sounds transmitted from the participants. The management system 208 is further configured to communicate with client terminals, 1, 2 . . . and M associated with respective users through a network 216. The network 216 may include one or more of the Internet, TV broadcasting network, satellite communication network, local area network (LAN), wide area network (WAN), personal area network (PAN), and other communication networks and associated protocols. The client terminals 1, 2 . . . and M may include smart phones, iPad®, tablets and other mobile devices or TV sets or desktops. Each client terminal has a screen and a speaker to reproduce the images and sounds that have been transmitted from a participant and processed by the management system 208. The transmission and playing back of the images and sounds may be handled by a TV broadcasting system or a computer, or an application that is configured to run on smart phones, laptops, tablets or other mobile devices. Various functions are performed by the management system 208 based on algorithms associated with a processor, e.g., CPU therein. The images and sounds received from the participants may be shown to the users real-time. Alternatively, they may be stored in a computer memory of the management system 208 and released later for later showing or downloading at the time the user specifies.

To further enhance the viewing experience, the present system includes a computer program to provide a game based on a process of an activity that the user decided to view. A process of an activity herein can be a progress of an activity. In general, “racing” refers to sports for competing in speed among racers, such as field athletes and swimmers, or those involving motorcycles, cars, bicycles, boats, aircrafts, horses, skis, skates, skateboards, sleighs, wheelchairs, yachts, and other vehicles or animals associated with respective racers. Other sports or activities, not categorized as “racing”, involve individual accomplishments during a process of an activity by using metrics such as certain types of goals or milestones. This is different from competing in speed among a plurality of racers in one race. The present game is based on a process of an activity, such as a goal and/or one or more milestones achievable by a participant in the activity that the user selected to view, thereby not involving competition in speed among racers/participants in one race. Examples of such sports or activities involving individual accomplishments based on a goal and/or one or more milestones may include, but not limited to, the following:
Mountain climbing, wherein the mountain climber reaches 50% of the elevation at about 6 hours from the base; and reaches the top of the mountain at about 10 hours from the camp at the 50% of the elevation.
Deep sea exploration, wherein the deep sea diver reaches the bottom of the ocean at about 30 minutes; and takes photos of three different deep-sea creatures within another 30 minutes.
Spacewalk, moving in zero-gravity, walking on the moon and other space activities, wherein the astronaut works to replace a mechanical part in a solar panel within 30 minutes.
Sports, such as soccer, football, boxing, fencing, wrestling, karate, taekwondo, tennis and others, wherein the player scores a goal or a first point within 30 minutes from the start.
Firefighting, wherein the firefighter puts out the fire within 30 minutes.
Open heart surgery, wherein the surgeon cuts open the patient's breastbone to expose the heart within 30 minutes from the injection of general anesthesia, replaces a heart valve within another 30 minutes, completes sternal plating within another 30 minutes, and completes the entire surgery at about 2 hours from the start.
Cooking, wherein the chef or amateur cooks an appetizer within 20 minutes, a main dish within 30 minutes, and to dessert within 20 minutes.
   The participant in each activity may have his/her own goal and/or one or more milestones prior to starting the activity, although he/she may or may not actually accomplish his/her original goal and/or one or more milestones. However, the participant would at least try to pursue the activity with his/her predetermined plan. In the game that can be configured based on the present system, the user who selected a participant in a particular activity can input, prior to receiving the images and sounds from the participant, the user's predictions related to the goal and/or one or more milestones expected of the participant in the activity. The user's predictions will be compared against actual accomplishments by the participant for determining win/loose of the game, whereby the user can virtually experience the activity with the perspective of the participant with the uncertainty and excitement with respect to whether the participant can accomplish the expected goal and/or one or more milestones.

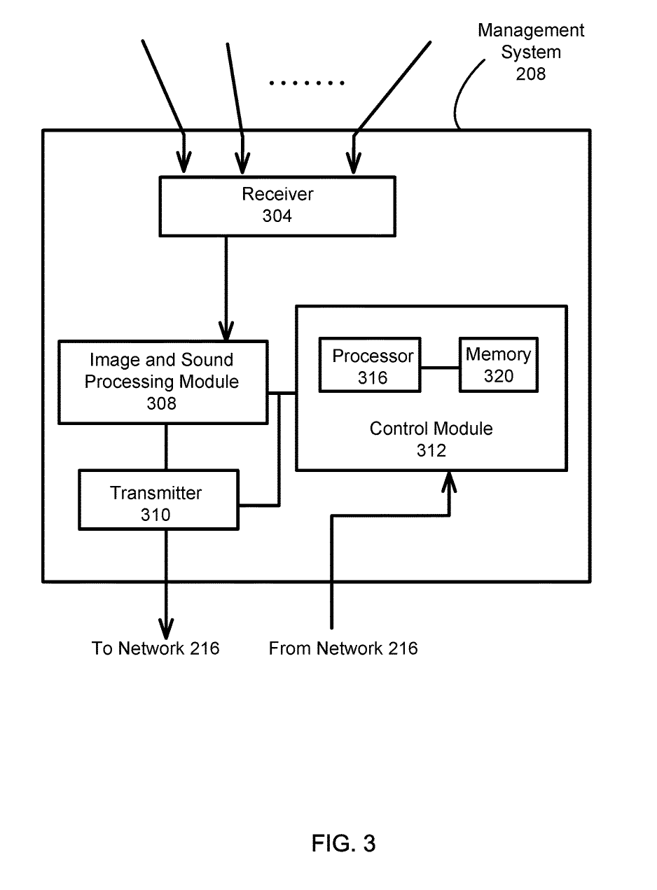

FIG. 3 is a block diagram illustrating the management system 208 that controls transactions, receiving and transmitting the images and sounds, executing the game and various other functions. The signals transmitted from the participants are received by a receiver 304. The receiver 304 may include an antenna and other RF components for analog-to-digital conversion, digital-to-analog conversion, low noise amplification, digital signal processing, etc. to receive the signals. Any receiver technologies known to those skilled in the art can be utilized for implementation of the receiver 304 as appropriate. The received signals are sent to an image and sound processing module 308, where the images and sounds are processed and prepared for transmission to the client terminals. Examples of image processing techniques may include software algorithms to process digital images based on information contained in pixels. Existing and/or modified techniques can be used for compression, decompression, layering, sizing, sharpening, orienting, lens-correcting, selecting, merging, contrasting, brightening, cropping, and various other rapid processing of the images, i.e., visual signals. For example, the images with different perspectives captured by two or more cameras of the participant may be processed for the user to experience the 3D effect. In another example, blurred or rapidly fluctuating images due to camera shaking may be corrected to be viewed without causing discomfort to the user. Examples of sound processing techniques may include hardware components, such as filters, as well as software algorithms to process sound waves in analog and/or digital domains. Existing and/or modified techniques can be used for equalization, filtering, frequency modulation, noise reduction, time-stretching, compression and various other rapid processing of the sounds, i.e., audio signals. For example, a loud noise, such as the roaring sound of a vehicle, may be reduced to a comfort level. In another example, the sounds from different audible perspectives captured by two or more microphones of the participant may be processed for the user to experience the stereophonic effect. Any image and sound processing technologies known to those skilled in the art can be utilized for implementation of the image and sound processing module 308 as appropriate. The images and sounds may simply be processed to fit in certain sizes or lengths, or formatted properly for end client terminals. The processed images and sounds may be stored in a memory and transmitted out later, or sent directly to a transmitter 310 configured to transmit out the processed images and sounds to the client terminals through the network 216. The transmitter 310 includes electronic components and modules necessary for RF transmission, such as power amplification, oscillation, modulation, impedance matching, etc. Any transmitter technologies known to those skilled in the art can be utilized for implementation of the transmitter as appropriate. Alternative to having a receiver and a transmitter separately, a combined transceiver may be configured for the present system 208.

The management system 208 further includes a control module 312, which may include a processor 316 for controlling hardware and software and a memory 320 for storing data, information and programs. Examples of such stored data include predetermined data and/or acquired data, e.g., information pertaining to users. The data can be updated or retrieved as needed. The memory 320 may store one or more computer programs having computer executable instructions for performing functions and tasks. The processor 316 executes a computer program having computer executable instructions. The images and sounds received from the participants may be stored in a database in the memory 320 before or after being processed by the image and sound processing module 308, and may be configured to be released real-time or later for later showing or downloading at the time the user specifies. The real-time showing can be configured with modern-day technologies, with a minor, tolerable time lag due primarily to the image and sound processing at the image and sound processing module 308.

The control module 312 is configured to receive input information that the users input at the respective client terminals and transmitted through the network 216. A prompt page may be configured for the users to input necessary information. The input information pertains to the user, including an ID of the user, his/her choice of the payment method (credit card, PayPal®, money order, etc.), his/her credit card number if the credit card payment is chosen, and other account information. The user makes the payment to view the real-time or later showing or to download the stored video of the activity he/she chooses. The user is prompted to select or specify an activity that he/she wishes to view and a participant in the activity whom he/she wishes to get connection with for receiving the images and sounds as perceived by the selected participant. One or more activities may be prepared for users to select from, each activity involving one or more participants. At least one of the one or more participants in each activity may be arranged to capture and transmit images and sounds during the activity, for users to select from. As illustrated in the example in FIG. 1, one or more devices, each including at least one camera and at least one microphone can be provided with the participant. Alternatively, at least one camera and at least one microphone separately can be provided with the participant. In this way, the user can share the common experience with the actual participant via the images and sounds captured by the cameras and microphones placed in the proximity of the participant's eyes and ears. The participant whom a user selects may generally be his/her favorite player, performer, athlete, or other professionals, or his/her friend. Therefore, the present system enables a user to virtually experience the activity with the perspective of the actual participant, with whom the user may feel bonding, which can enhance the enjoyment.

In addition to the information necessary for viewing, the user may be asked which activity is his/her favorite. Another personal preference, such as his/her favorite participant, may also be added. Such personal information pertaining to users can be stored in the memory 320, and may be utilized for the operation entity 202 to plan the types of and participants in activities in the future to improve customer satisfaction. The information from users may be stored in the memory 320 and updated when any of the users change his/her account information, activity of choice, favorite participant, favorite activity, or any other user information.

Upcoming activities and schedules may be sent in advance by the control module 312 to the client terminals. The users may request to receive such information via emails or text messages. Alternatively, such information can be broadcast via audio/visual media, e.g., social network media. The schedule may list the names or IDs of the participants participating in the upcoming activities so that the use can select the activity that his/her favorite, participant is scheduled to pursue. The fee for real-time viewing, later viewing or downloading may be a flat rate. Prior to the viewing or downloading, the input information including the account information and the choice of an activity and a participant is obtained by the control module 312 from the user as inputted at the client terminal. Payment can be made using the payment method that the user specified as part of the account information. Based on the user information, the control module 312 is configured to control the transmission of the processed images and sounds of the selected activity, as captured by the selected participant, to the client terminal of the user who selected the specific activity and the participant.

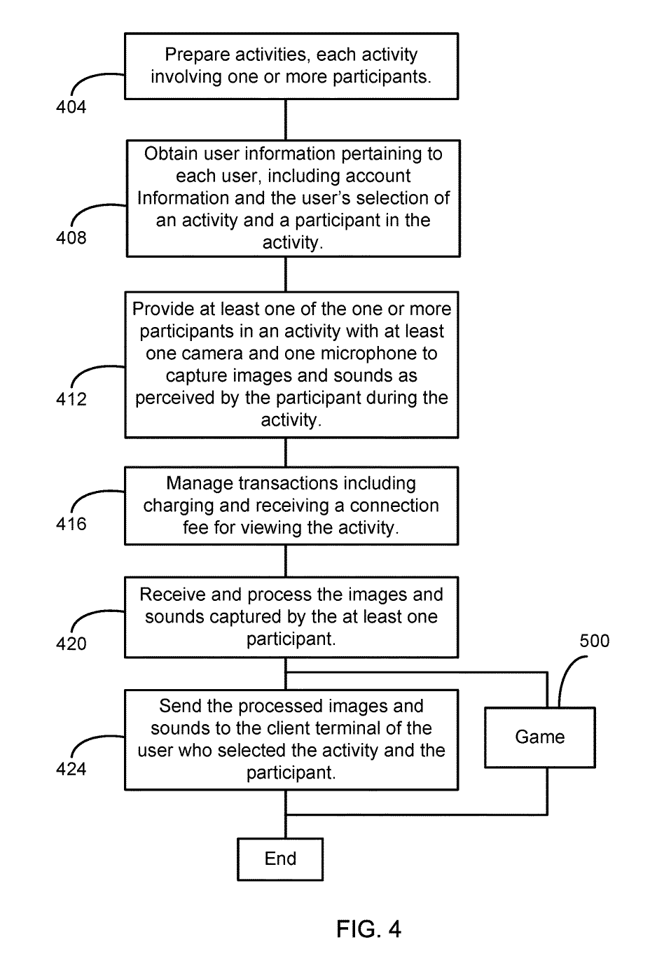

FIG. 4 is a flowchart illustrating a method of providing a user with super-reality entertainment including a. game based on a process of an activity selected by the user to view. A process of an activity herein can be a progress of an activity. Multiple activities can be planned; and a plurality of users can be entertained through the present system of FIG. 2 including the operation entity 202, management system 208, network 216 and a plurality of client terminals 1, 2 . . . and M that the users are respectively associated with. The order of steps in the flowcharts illustrated in this document may riot have to be the order that is shown. Some steps can be interchanged or sequenced differently depending on efficiency of operations, convenience of applications or any other scenarios. In step 404, one or more activities are prepared by the operation entity 202, each activity involving one or more participants. For example, the operation entity 202 decides on the types of activities to pursue, scheduling an activity to take place at a certain time and date, selecting a place that is proper for pursuing an activity, etc. Furthermore, the preparation in step 404 may include hiring or contracting with people who can actually participate in the activities, for example, an experienced mountain climber for mountain climbing 204-1, a professional boxer for boxing 204-2, . . . and a diver with a biology background for deep sea exploration 204-N, as illustrated in FIG. 2. The preparation may further include paying for expenses to pursue the activities, such as travel expenses and equipment purchase/rental fees, in addition to paying wages to the participants and supporting staff. In step 408, user information pertaining to each user is obtained, via, for example, a prompt page for inputting the information on a screen of the client terminal that the user is using. The input information may include account information, such as an ID of the user, his/her choice of the payment method (credit card, PayPal®, money order, etc.), his/her credit card number if the credit card payment is chosen, and so on. The input information may further include which activity and which participant in the activity are selected by the user. The input information may further include the user's favorite activity, favorite participant, and other personalized information. Such information pertaining to each user may be stored in the memory 320 in FIG. 3 of the management system 208 for reference. It is preferable that the operation entity 202 prepares the activities and hires or contracts with the participants that users would likely select. In the subsequent steps in the flowchart in FIG. 4, an example for the case of pursuing one activity is shown; however, it should be understood that the similar steps can be followed with respect to multiple activities in parallel or at different times. In step 412, at least one of the one or more participants is provided with at least one camera and one microphone. These devices can be attached to the proximity of the participant's eyes and ears so as to capture images and sounds as perceived by the participant during the activity. These devices may be attached to the face or head of the participant directly, or to a head gear, helmet, hat, headband, goggles, glasses or other item that the participant wears. In step 416, the transaction is managed, including charging and receiving a connection fee for viewing real-time or downloading the activity video for later viewing. The fee can be paid through the payment method that the user specified. In step 420, the images and sounds captured by the device or devices attached to the at least one participant in the activity are received by the receiver 304 and processed by the image and sound processing module 308 in FIG. 3. The images and sounds may simply be processed to fit in certain sizes or lengths, or formatted properly for end client terminals. In another example, the images with different perspectives captured by two or more cameras of the participant may be processed for the user to experience the 3D effect. In another example, blurred or rapidly fluctuating images due to camera shaking may be corrected to be viewed without causing discomfort to the user. In another example, a loud noise, such as the roaring sound of a vehicle, may be reduced to a comfort level. In another example, the sounds from different audible perspectives captured by two or more microphones of the participant may be processed for the user to experience the stereophonic effect. In step 424, the processed images and sounds captured by the at least one participant are sent to the client terminal of the user who selected the activity and the participant in the activity. The images and sounds may be stored in the memory 320, before or after the processing at the image and sound processing module 308, and released real-time or later for later showing or downloading at the time the user specifies. The real-time showing can be arranged by transmitting out the processed images and sounds directly without storing, but may experience a minor time lag due primarily to the image and sound processing at the image and sound processing module 308.

As mentioned earlier, to further enhance the viewing entertainment, the present system can be configured to provide a game based a process of the activity that the user selected to view. The computer program to execute the game may comprise an application installed with the user's client terminal, a software algorithm installed with the control module 312, or both working collaboratively with each other. The computer program has computer executable instructions to perform steps to provide a user with the game. The application can be designed to perform tasks, executable with an operation system in a client terminal, such as a smart phone, a tablet, a laptop computer and other mobile devices, as well as in a TV system, a desktop computer, and other immobile devices. The application can be downloaded from a site associated with the server including the management system 208 through the Internet and placed in the client terminal, distributed directly from the distributer/developer of the application, or placed externally to the client terminal, for example, in the cloud computing environment. The application installed with a client terminal may have at least a user interface enabling the user to input information, a communication unit for receiving and transmitting information from/to the management system 208, and a display control for displaying the information. The software algorithm installed with the control module 312 may be configured to perform calculations, decision makings, control of information flow, and various other takes related to carrying out the game in collaboration with the application installed at each client terminal. Alternatively, all the functions and tasks may be configured to be performed by an application installed with the user's client terminal, or by a software algorithm installed with the control module 312. In the case where all the instructions for executing the game are included in the application, the control nodule 312 is configured to interact with the application for controlling input and output flow, data exchange and various other functions associated with the game playing.

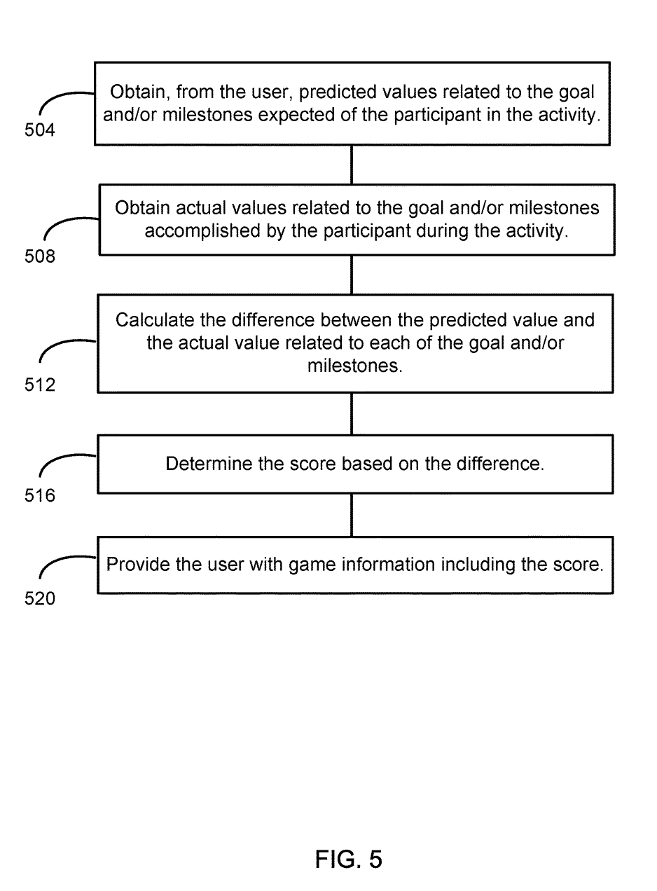

In FIG. 4, as an example, a game process 500 is shown to be carried out simultaneously with the transmission of the processed images and sounds from the system 208 to a client terminal associated with the user who selected the activity and the participant in the activity. The other tasks and functions can also be performed in the background while the game 500 is being played. FIG. 5 is a flowchart illustrating steps of executing the game 500 based on the computer executable instructions. As mentioned earlier, the computer program to execute the game may comprise an application installed with the user's client terminal, a software algorithm installed with the control module 312, or both configured to work with each other collaboratively. In FIGS. 4 and 5, the game process played by one user is illustrated; however, the present game can be played individually by a plurality of users associated with respective client terminals, simultaneously or at different times. Each of the plurality of users selected a participant in the activity to get connection with for receiving the images and sounds from the selected participant. After step 408, wherein the user selected an activity to view as well as to participant in the activity to get connected with for receiving the images and sounds captured by the participant during the activity, in step 504, predicted values related to the goal and/or one or more milestones expected of the participant in the activity are obtained from the user prior to viewing the activity. The computer program may be configured to obtain the predicted values, by displaying, for example, a prompt page for the user to input such information at the client terminal. In an example of mountain climbing, the user may input the first predicted time when the mountain climber reaches 50% of the elevation, e.g., 6 hours from the base, and the second predicted time when the mountain climber reaches the summit, e.g., 10 hours from the camp located at the 50% of the elevation. The mountain climber may have planned his/her own goal and/or one or more milestones prior to starting the mountain climbing, although he/she may or may not actually accomplish his/her original goal and/or one or more milestones, due to unexpected bad weather or difficulty, for example. However, the mountain climber would at least try to pursue the mountain climbing with his/her predetermined plan. The user's predictions will be compared against actual accomplishments by the mountain climber for determining win/loose of the game. The predicted values inputted by the user may be sent to the control module 312 and stored m the memory 320, or may be stored in the memory of the client terminal by the application. In step 508, actual values related to the goal and/or one or more milestones during the activity are obtained. For example, in the activity of mountain climbing, the location and time of the mountain climber can be obtained from the GPS. Alternatively, the mountain climber can record the data using his altitude meter and clock, and send the same to the management system 208. The control module 312 may be configured to receive and store the location and time data in the memory 320, or directly send the data to the client terminal. In step 512, the difference between the predicted value and the actual value related to each of the goal and/or one or more milestones is calculated. In step 516, the score is determined based on the difference. For example, it is determined whether the difference is within a predetermined range for each of the goal and/or one or more milestones; and if it is within, one point is given, whereas if it is outside the range zero point is given. Alternatively, the predetermined range may have two or more sub-ranges for different scoring. For example, if the difference is within a first sub-range, two points are given; if the difference is between the first sub-range and a second sub-range that is wider than the first sub-range, one point is given; and if the difference is outside the second sub-range, zero point is given. This and other scoring variations are possible, and can be implemented in the computer program. These ranges and corresponding points to be given may be predetermined by the operation entity 202, by the participant, by the computer program based on the history of the particular activity, or by any other suitable method, and may be stored in the memory 320 prior to starting the game. In step 520, game information is provided to the user, including the score. The score can be sent to the user separately from or simultaneously with the transmission, in step 424 of FIG. 4, of the processed images and sounds captured by the participant during the activity. The points may be accumulated by playing multiple games, and made redeemable. For example, the user may use a predetermined number of points to exchange with the next connection fee to be paid to view an upcoming activity and/or play the game. Alternatively, sponsors or advertisers may prepare goods, services or coupons that can be exchanged with a predetermined number of points.

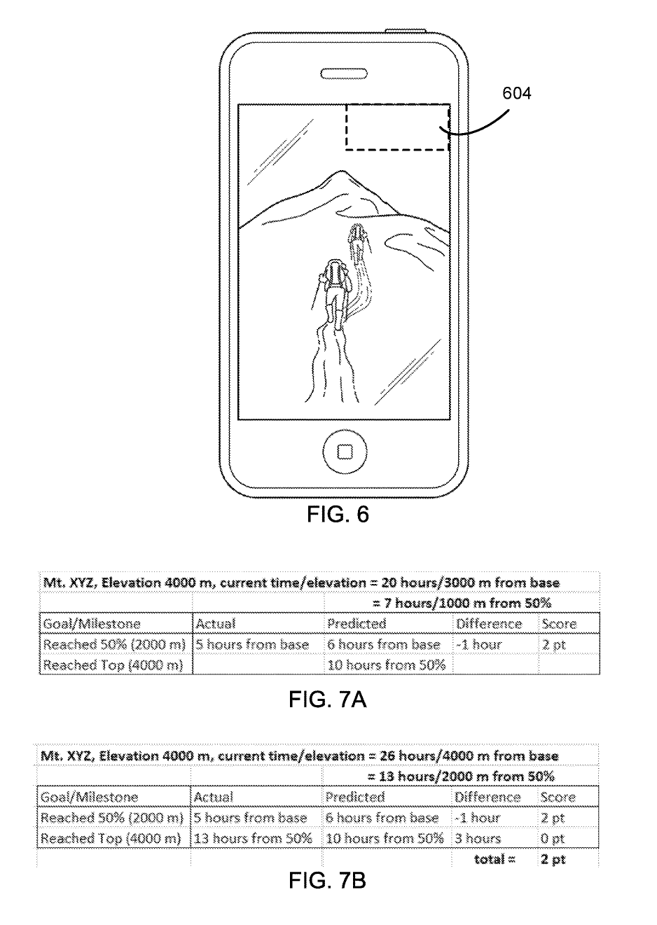

FIG. 6 illustrates an example of a view displayed on a screen of a client terminal, wherein the score of the game and the processed images and sounds are simultaneously transmitted to the user who selected the activity and the participant to get connection with for receiving the images and sounds as perceived by the participant. The score, actual values and predicted values related to each of the goal and/or one or more milestones are overlaid with the images and sounds. A view from the perspective of the mountain climber while climbing a snow-capped mountain as illustrated in FIG. 1B is transmitted to the smart phone in the example of FIG. 6. The mountain climber is attached with one or more devices as illustrated in FIG. 1A, which are configured to capture the images and sounds during the mountain climbing as perceived by the mountain climber. The images and sounds are received by the receiver 304, processed by the image and sound processing module 308, and transmitted through the network 216 to the client terminal of the user who selected to view the mountain climbing activity with the perspective of the mountain climber he/she selected, in the present example, the game information 604 is displayed in the right upper corner of the frame of the images, showing the progress of the mountain climbing, such as the actual and predicted times to reach the 50% elevation (milestone) and the top (goal), along with the score of the game. The display area for the game information 604 can be anywhere in the viewable screen and can be zoomed in or out. Alternatively, the game information 604 can be displayed in a separate page by a click or other command from the user.

FIGS. 7A and 7B illustrate examples of the game information 604 for the case of climbing Mt. XYZ with the elevation 4000 m. The user has inputted his/her predicted values, which are displayed as part of the game information 604. In FIG. 7A, the mountain climber is currently at 3000 m from the base after 20 hours, equivalently, 1000 m from 50% (2000 m) of the elevation after 7 hours, wherein it took 5 hours to reach the 50% elevation and be spent 8 hours to rest in the camp located at the 50% elevation. On the other hand, the user predicted that he would reach the 50% elevation after 6 hours. The difference, i.e., actual−predicted=5 hours−6 hours=−1 hour, is calculated and shown. The difference is within the range between −2 hours and 2 hours predetermined for the milestone; thus, the user scored 2 points for sufficiently accurately predicting the time it takes for the mountain climber to reach the 50% of the elevation. In FIG. 7B, the mountain climber has just arrived at the top (4000 m) after 26 hours from the base, wherein it took 5 hours to reach the 50% elevation and he spent 8 hours to rest in the camp located at the 50% elevation. As such, it took 13 hours to reach the top from the camp located at the 50% elevation. On the other hand, the user predicted that he would reach the top after 10 hours from the 50% elevation. The difference, i.e., actual−predicted=13 hours−10 hours=3 hours, is calculated and shown. The difference is outside the range between −2.5 hours and 2.5 hours predetermined for the goal; thus, the user scores 0 point for underestimating the time it takes for the mountain climber to reach the top. Therefore, the user obtained the total score of 2 points by playing the present game.

The computer program for executing the game may be further configured to reproduce the images and sounds at the client terminal with a proper format and/or control options for the user to control the images and sounds. For the game based on an activity that may take a long time, such as mountain climbing, the computer program may be configured or built-in functions of the client terminal can be utilized to interrupt and later resume the viewing and the game. These and other functions, known to those skilled in the art, can be implemented in the system and the computer program, allowing users to comfortably enjoy the super-reality entertainment and associated game based on a process or progress of an activity.

While this document contains many specifics, these should not be construed as limitations on the scope of an invention or of what may be claimed, but rather as descriptions of features specific to particular embodiments of the invention. Certain features that are described in this document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be exercised from the combination, and the claimed combination may be directed to a subcombination or a variation of a subcombination.

What is claimed is:

1. A method of providing a user with entertainment, for which one or more activities are prepared, each activity in one or more participants, the method comprising:
obtaining user information pertaining to the user, the user information including the user's selection of an activity and a participant in the activity;
receiving images and sounds captured by at least one camera and one microphone attached to the participant, as perceived by the participant during the activity;
processing the images and sounds;
sending the processed images and sounds to a client terminal associated with the user who selected the activity and the participant in the activity; and
providing the user with a game based on a process of the activity.

2. The method of claim 1, wherein the providing the user with the game based on the process of the activity comprises:
obtaining, from the user prior to the sending the processed images and sounds to the client terminal associated with the user, predicted values related to a goal, one or more milestones, or a combination thereof expected of the participant in the activity;
obtaining actual values related to the goal, the one or more milestones, or a combination thereof accomplished by the participant during the activity;
calculating a difference between the predicted value and the actual value related to each of the goal, the one or more milestones, or a combination thereof:
determining score based on the difference; and
providing the user with game information including the score.

3. The method of claim 2, wherein
the score is represented by zero or more points that are redeemable.,

4. The method of claim 2, wherein
the game information includes the process of the activity including the score, the actual values and the predicted values and is displayed with the processed images and sounds on the client terminal associated with the user.

5. The method of claim 1, further comprising:
sending to the user, prior to the obtaining the user information, a schedule listing one or more activities and names or IDs of one or more participants in each of the activities that are upcoming.

6. The method of claim 1, wherein
the user information further includes personal information including the user's favorite activity and favorite participant.

7. The method of claim 1, wherein
the at least one camera and one microphone are attached to a face or a head of the participant, or to a head gear, a helmet, a hat, a headband, goggles, glasses or other head or face mounted item that the participant wears.

8. The method of claim 1, wherein
the processing the images and sounds comprises correcting blurred or rapidly fluctuating images due to camera shaking.

9. The method of claim 1, wherein
the processing images and sounds comprises processing sounds from different audible perspectives to generate a stereophonic effect.

10. The method of claim 1, wherein
the processing images and sounds comprises processing images with different perspectives to generate a three-dimensional effect.

11. The method of claim 1, wherein
the sending the processed images and sounds comprises releasing real-time or releasing at a time the user specifies the processed images and sounds.

12. A system for providing a user with entertainment, for which one or more activities are prepared, each activity involving one or more participants, the system comprising:
a control module configured to obtain user information pertaining to the user, the user information including the user's selection of an activity and a participant in the activity, and to include at least part of or interact with a computer program for providing the user with a game based on a process of the activity;
a receiver for receiving images and sounds captured by at least one camera and one microphone attached to the participant, as perceived by the participant during the activity; and
an image and sound processing module configured to process the images and sounds; and
a transmitter configured to send the processed images and sounds to a client terminal associated with the user who selected the activity and the participant in the activity.

13. The system of claim 12, wherein the computer program for providing the user with the game based on the process of the activity comprises computer executable instructions for performing:
obtaining, from the user prior to sending the processed images and sounds to the client terminal associated with the user, predicted values related to a goal, one or more milestones, or a combination thereof expected of the participant in the activity;
obtaining actual values related to the goal, the one or more milestones, or a combination thereof accomplished by the participant during the activity;
calculating a difference between the predicted value and the actual value related to each of the goal, the one or more milestones, or a combination thereof;
determining score based on the difference; and
providing the user with game information including the score.

14. The system of claim 13, wherein
the game information includes the process of the activity including the score, the actual values and the predicted values and is displayed with the processed images and sounds on the client terminal associated with the user.

15. The system of claim 12, wherein
the control module is further configured to send to the user, prior to obtaining the user information, a schedule listing one or more activities and names or IDs of one or more participants in each of the activities that are upcoming.

16. The system of claim 12, wherein
the control module comprises a memory for storing at least the processed images and sounds and the user information.

17. The system of claim 12, wherein
the client terminal is a TV system, a desktop computer or other immobile device, or a smart phone, a laptop computer, a tablet or other mobile device.

18. The system of claim 13, wherein
the image and sound processing module is configured to perform one or more of operations comprising:
correcting blurred or rapidly fluctuating images due to camera shaking;
reducing a loud noise to a comfort level;
processing sounds from different audible perspectives captured by two or more microphones to generate a stereophonic effect; and
processing images with different perspectives captured by two or more cameras to generate a three-dimensional effect.

19. The system of claim 12, wherein
the computer program comprises an application installed with the client terminal associated with the user, a software algorithm installed with the control module, or both configured to work collaboratively with each other.
